(19) United States
(12) Patent Application Publication
(10) Pub. No.: US 2011/0106113 A1
(43) Pub. Date: May 5, 2011

(54) SYSTEM AND METHOD FOR HERNIA MESH FIXATION

(75) Inventors: Ali Tavakkolizadeh, Brookline, MA (US); Michael Eilenberg, Port Washington, NY (US); Jessica Galie, Cambridge, MA (US); Megan Roberts, Cambridge, MA (US); Martin Culpepper, Danvers, MA (US)

(73) Assignees: THE BRIGHAM AND WOMEN'S HOSPITAL, INC., Boston, MA (US); MASSACHUSETTS INSTITUTE OF TECHNOLOGY, Cambridge, MA (US); HARVARD MEDICAL SCHOOL, Boston, MA (US)

(21) Appl. No.: 12/669,005

(22) PCT Filed: Jul. 14, 2008
(86) PCT No.: PCT/US08/08589

§ 371 Date: Nov. 23, 2010

Related U.S. Application Data
(60) Provisional application No. 60/959,343, filed on Jul. 13, 2007.

(51) Int. Cl. A61B 17/00

(52) U.S. Cl. 606/151

(57) ABSTRACT

The invention includes a surgical fastener and associated deployment system and method that overcomes the drawbacks of prior art surgical mesh fixation devices. The surgical fastener and deployment system may be used to fixate a surgical mesh material to the abdominal wall for the purpose of hernia repair. In accordance with one embodiment, the fastener may include an anchor head comprising a bi-pyramid framework. The anchor head is preferably made from a highly deformable and biocompatible material that withstands high flexural strain within an oscillatory environment. The anchor head may be provided in an elongate, undeployed configuration, and then expanded during deployment into a second, generally planar configuration. The anchor head may be biased to expand into the generally planar configuration from the undeployed configuration in a variety of manners.

### CROSS-REFERENCE TO RELATED APPLICATION

This application claims priority to U.S. Provisional Patent Application Ser. No. 60/959,343, Filed Jul. 13, 2007. This patent application is incorporated by reference herein in its entirety.

### BACKGROUND OF THE INVENTION

1. Field of the Invention

The present invention relates to systems and methods for attaching a prosthetic device to the surface of tissue, and more particularly, to the application of mesh to cover a hernia defect as well as devices for applying such a mesh and holding such mesh in a desired position.

2. Description of Related Art

The anterior abdominal wall is comprised of a muscle layer, surrounded by strong connective tissue known as fascia. Adipose tissue (fat) and dermal layers (skin) are located on the outside of the muscle layer. A weakness in the abdominal wall, for example caused by a former surgical incision, may allow the internal organs to pass through, causing a hernia. Hernias are relatively common and may cause pain or strangulation of the bowel, in which blood flow to the tissue is restricted. Such hernias often need to be repaired.

Many methods of hernia repair are known. Among the most popular is the use of a mesh barrier placed on the inside of the abdominal wall to cover the defect. This procedure can be accomplished through open surgery, however minimally invasive surgery is becoming increasingly popular as a new approach to treat this condition.

The minimally invasive laparoscopic surgical techniques typically require only a few small incisions (0.5-1.5 centimeters) in the abdomen, instead of a larger incision typical of open surgery. A trocar (i.e., a tube-shaped port which typically has a 5-12 millimeter internal diameter) is inserted into each incision. The abdomen is then inflated with insufflation gas (e.g., carbon dioxide), and then a small camera and surgical tools are advanced through the trocars. The image from the camera is typically projected on a monitor in the operating room, allowing the surgeon to see the inside of the cavity and the extent of the defect in the abdominal wall. Laparoscopic tools are generally designed with a long wand-like distal end that is inserted into the cavity through the trocar. The wand-like distal end is then positioned manually by the surgeon and may be activated, for example, by the squeeze of a trigger or other suitable means.

For cases of laparoscopic ventral hernia repair, the surgeon first identifies the hernial defect before cutting the mesh to be about 3-5 centimeters longer in diameter than the size of the hole itself. The mesh is then inserted into the abdominal cavity through a trocar, and secured to the anterior abdominal wall in such a fashion that it covers the hernial defect. To provide a secure fixation of the mesh to the anterior abdominal wall, sutures are often used to secure the mesh to the abdominal wall. The sutures are placed on the mesh and then advanced through the abdominal wall until they are visible outside the abdominal wall. The sutures are then tied off against the abdominal wall. Generally, 4 or more sutures are used to fix the mesh to the abdominal wall, depending on the size of defect. Tacks are then typically applied near the perimeter of the mesh to fix the mesh to the abdominal wall. The tacks are placed at close intervals, preventing the bowels (or other organs) from passing between the mesh and the abdominal wall. Such tacks come in several varieties and may be made of metal or absorbable materials; typical examples can be found, for example, in U.S. Pat. No. 6,036,701, U.S. Pat. No. 5,904,696, and U.S. Pat. No. 6,837,893 A1. Each of these patents is incorporated by reference herein in its entirety.

The laparoscopic method for repairing hernias may cause several problems. For example, transfascial sutures can often cause excessive post-operative pain. Specifically, internal forces exerted on the mesh are typically transferred to the muscle layer through these sutures. The sutures, in turn, concentrate these forces causing pain. Moreover, sutures have relatively low compliance compared to abdominal tissues, and therefore sutures may “pinch” when the muscle tissue contracts, similarly causing irritation to surrounding tissue. Furthermore, metal tacks (as described above) may occasionally dislodge from the abdominal wall, permitting them to irritate other tissue as they move within the body. Without the fasteners to hold the mesh in place, the mesh may come loose. These events may lead to additional complications, and possibly additional surgery.

Given the problems that are associated with current techniques of securing meshes, it is desirable to have a fastener and associated delivery system capable of penetrating all fascial layers of the abdominal wall, securing the mesh, and withstanding the internal forces of the body without patient discomfort and without the risks of fastener disengagement. It is also desirable that the fastener be delivered by way of laparoscopic techniques with minimal damage to surrounding tissues. It would also be advantageous to have a fastener that complies with surrounding bodily tissues. The present invention provides a solution for these problems.

### SUMMARY OF THE INVENTION

Advantages of the present invention will be set forth in and become apparent from the description that follows. Additional advantages of the invention will be realized and attained by the methods and systems particularly pointed out in the written description and claims hereof, as well as from the appended drawings.

To achieve these and other advantages and in accordance with the purpose of the invention, as embodied herein, the invention includes a surgical fastener and associated deployment system and method that overcomes the drawbacks of prior art surgical mesh fixation devices. The surgical fastener and deployment system may be used to fixate a surgical mesh material to the abdominal wall for the purpose of hernia repair.

In accordance with one embodiment, the fastener may include an anchor head comprising a bi-pyramid framework. The anchor head is preferably made from a highly deformable and biocompatible material that withstands high flexural strain within an oscillatory environment. The anchor head may be provided in an elongate, undeployed configuration, and then expanded during deployment into a second, generally planar configuration. The anchor head may be biased to expand into the generally planar configuration from the undeployed configuration in a variety of manners.

In accordance with an embodiment of a method of the invention, a fastener as embodied herein may be deployed using a delivery system as described herein. A distal end of a delivery device containing a fastener disposed in an undeployed state may be advanced to a location proximate the interior surface of the abdominal wall of a patient. The fastener may be deployed by advancing the fastener distally with respect to the delivery system, permitting the fastener to expand to a neutral, deployed state upon release from said introducer.

In accordance with a further aspect, the fastener may be disposed in an undeployed state within an introducer portion of the delivery system. In accordance with one embodiment, the introducer portion may be movably disposed within a main body portion of the delivery system. The distal end of the main body portion of the delivery system may be blunt to prevent damage to tissue, and the introducer portion may have a sharpened distal tip that may be advanced out of the distal end of the main body portion of the delivery system and advanced through fascia to permit transfascial fixation.

In accordance with a preferred embodiment, a sensing mechanism may be located proximate the distal end of the delivery system (e.g., at the distal end of the introducer portion) to detect the difference between muscle, fascial, and adipose tissues, to facilitate accurate transfascial placement of the surgical fastener. Once the fastener is deployed from the introducer, in accordance with one embodiment, a portion of the fastener may be collapsed (e.g., the anchor head portion) by application of a force to the fastener (e.g, applied by way of a suture or other filament embedded in the anchor head). When the fastener is approximately flush with the fascia in a generally planar state, a suture clip or other fastener may then be applied (e.g., to the filament on the interior surface of the abdominal wall) thus keeping the mesh against the interior surface of the abdominal wall. Surgical fastener systems made in accordance with the present disclosure are preferably compatible with commercial suture clip appliers. As will be understood by those of skill in the art, the fastener and the delivery device may be take on a variety of configurations within the spirit and scope of the present disclosure.

It is to be understood that the foregoing general description and the following detailed description are exemplary and are intended to provide further explanation of the invention claimed.

The accompanying drawings, which are incorporated in and constitute part of this specification, are included to illustrate and provide a further understanding of the method and system of the invention. Together with the description, the drawings serve to explain principles of the invention.

### BRIEF DESCRIPTION OF THE DRAWINGS

FIG. 1 is an illustration of an exemplary coordinate system {X, Y, Z} defined with respect to the body of a patient during an exemplary laparoscopic procedure.

FIG. 2(a) is an elevation view of a first representative embodiment of a fastener made in accordance with the invention in an elastically relaxed state.

FIGS. 2(b)-2(c) illustrate an exemplary method for manufacturing the fastener of FIG. 2(a).

FIG. 3 is a top view of the fastener of FIG. 2(a) in an elastically relaxed state.

FIG. 4 is a perspective view of the fastener of FIG. 2(a) in an elastically relaxed state.

FIG. 5 is an elevation view of the fastener of FIG. 2(a) in an elastically deformed state prior to deployment.

FIG. 6 is a top view of the fastener of FIG. 2(a) in an elastically deformed state prior to deployment.

FIG. 7 is an elevation view of the fastener of FIG. 2(a) in a deployed state.

FIG. 8 is a top view of the fastener of FIG. 2(a) in a deployed state.

FIG. 9 is an elevation view illustrating a filament (e.g. a suture) incorporated into an exemplary embodiment of a fastener made in accordance with the invention.

FIG. 10 is a top view illustrating a filament (e.g. a suture) incorporated into an exemplary embodiment of a fastener made in accordance with the invention.

FIG. 11 is an illustration of an exemplary embodiment of a delivery system made in accordance with the invention.

FIG. 12 is an illustration of a distal portion of an exemplary embodiment of a delivery system made in accordance with the invention.

FIG. 13 depicts schematic views illustrating exemplary wiring layouts for an electrical impedance sensor for the delivery system made in accordance with the invention.

FIG. 14 depicts schematic views illustrating exemplary optical fiber layouts for an optical sensor for the delivery system made in accordance with the invention.

FIG. 15 is an illustration of a distal portion of a delivery system made in accordance with the invention that depicts an exemplary arrangement of electrical impedance contacts.

FIG. 16 is an illustration of a distal portion of a delivery system made in accordance with the invention depicting an exemplary arrangement of optical fibers

FIG. 17 is an illustration of an exemplary sensor readout for a tissue depth gauge made in accordance with the invention.

FIG. 18 is an illustration of another exemplary sensor readout for a tissue depth gauge made in accordance with the invention.

FIGS. 19(a)-19(f) are schematic views illustrating the insertion and the locking of an exemplary fastener made in accordance with the invention between fascial and adipose tissues.

FIG. 20 is a schematic view of a deployed fastener in accordance with the invention.

FIG. 21 is an elevation view of a second representative embodiment of a fastener made in accordance with the invention in an elastically relaxed state.

### DETAILED DESCRIPTION OF PREFERRED EMBODIMENTS

Reference will now be made in detail to the present preferred embodiments of the invention, examples of which are illustrated in the accompanying drawings. The method and corresponding steps of the invention will be described in conjunction with the detailed description of the system.

Devices and methods provided in accordance with the invention may be used generally in surgical procedures. Such devices and methods are particularly advantageous in affixing mesh to tissue in the course of surgery to repair a hernia defect.

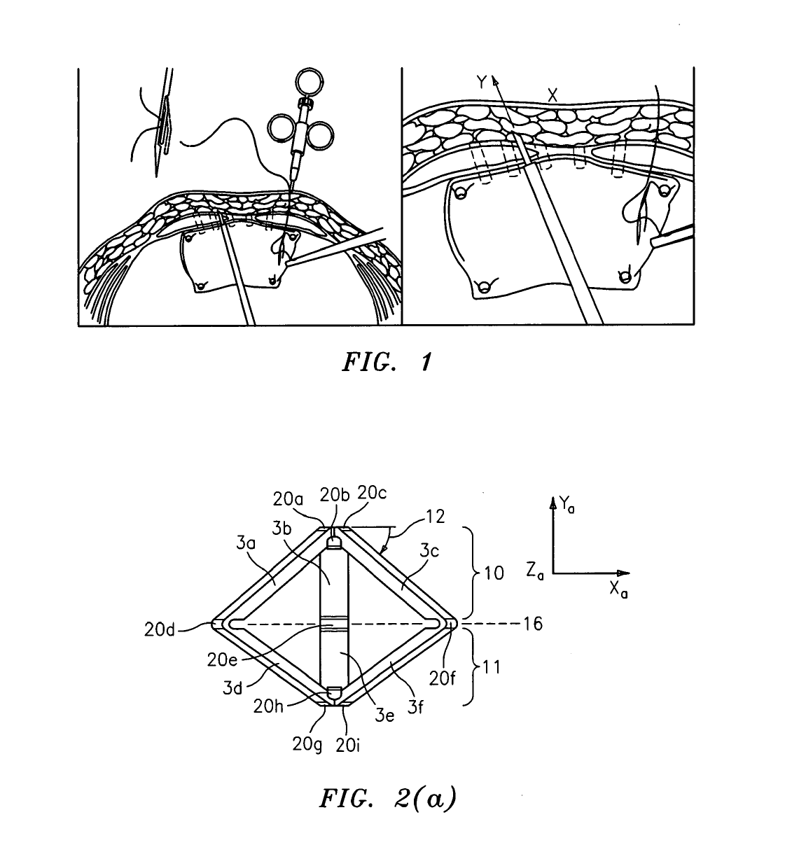

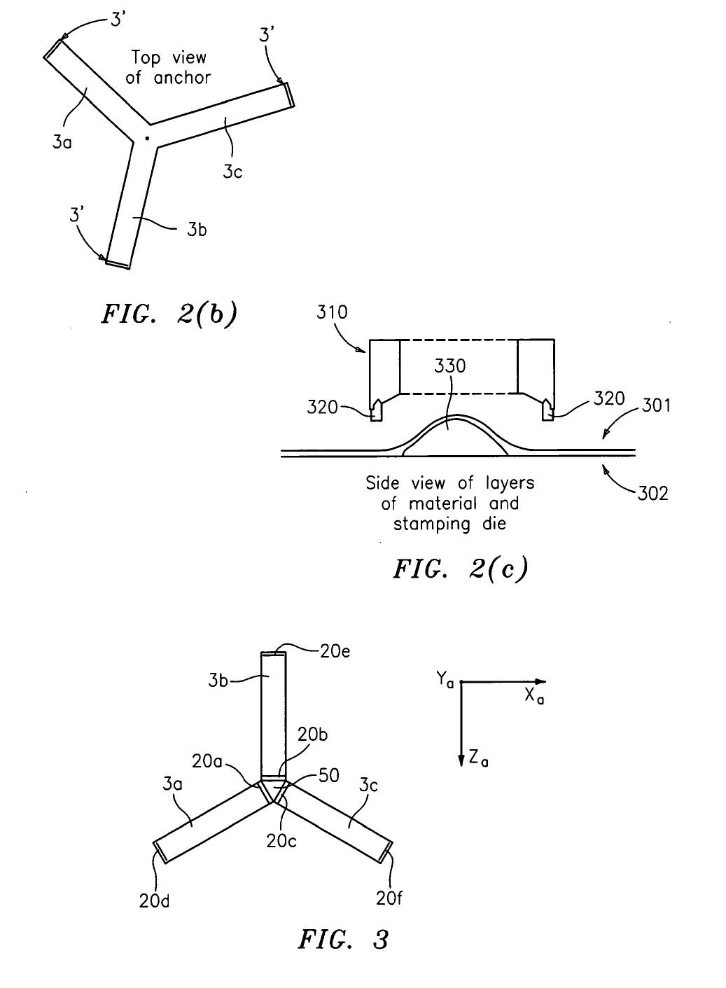

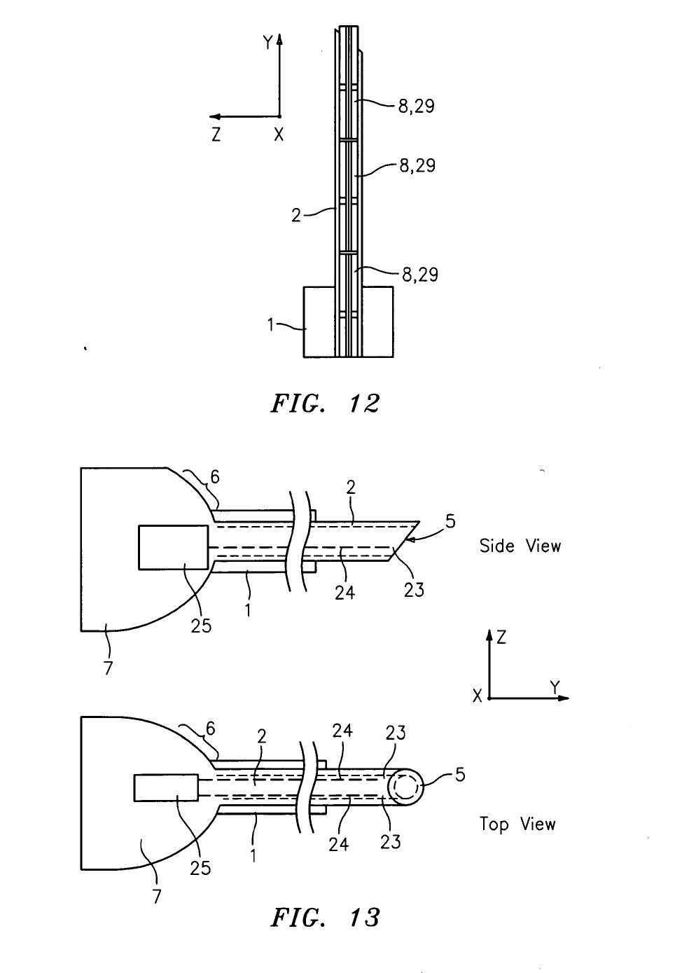

For purposes of illustration, and not limitation, exemplary embodiments of devices and methods provided by the invention are illustrated in FIGS. 2-21 herein. In accordance with a first embodiment, an improved fastener is provided that may be delivered using a delivery system.

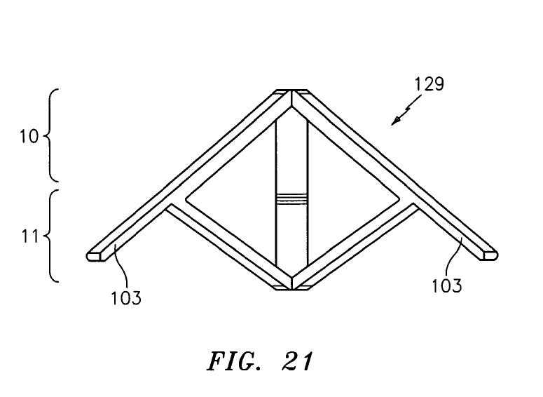

As embodied herein and as depicted in FIG. 2(a), the fastener includes an anchor head portion 29. Various views of the fastener of FIG. 2(a) are provided in FIGS. 3-12 and 19-20. A second representative embodiment of an anchor head 129 is depicted in FIG. 21 and described in detail below. In these drawings, an anchor head is depicted with three legs although it could include any suitable number of legs or struts (e.g., 2, 4, 5, 6, etc.). In accordance with the present disclosure, three struts are preferred.

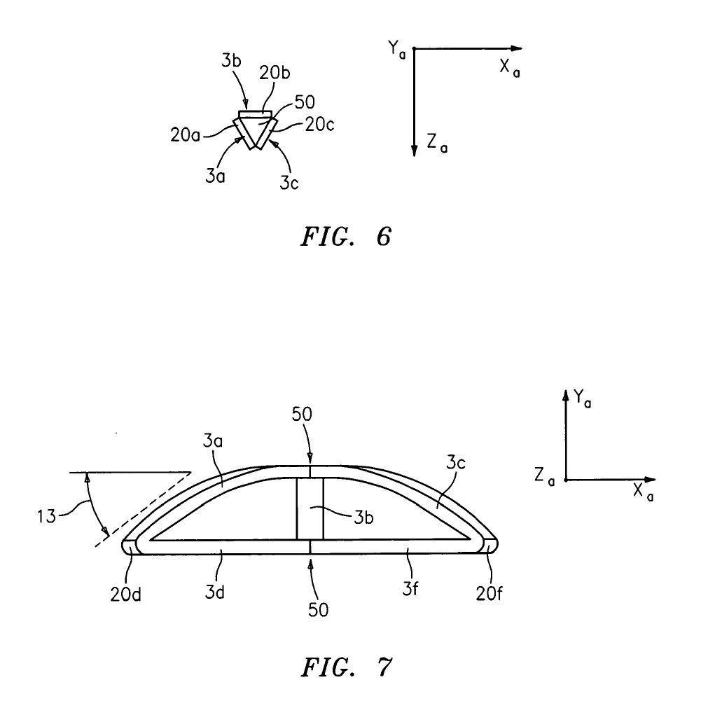

As depicted, the anchor head 29 includes three equally-spaced legs 3a-f whose ends are connected to form the framework of a deformable bi-pyramid, as shown, for example, in FIG. 2(a). The bi-pyramid comprises a polyhedron that is formed by joining a pyramid and its mirror image in a base-to-base arrangement. While it is shown in FIG. 6 that each leg (also referred to herein as a “strut”) 3a-f has a generally rectangular cross-sectional profile, other cross-sectional profiles may be employed (e.g., circular, triangular, elliptical, etc.) to modify the anchor for specific uses.

In accordance with a preferred embodiment, anchor head 29 is made from a durable and deformable biocompatible material, such as medical grade polyethylene or polypropylene. Examples of other materials that may be used to construct the anchor head 29 include, but are not limited to, nickel-titanium alloys (e.g., NITINOL®), other shape memory materials, silicone, polyurethane, polyethylene terephthalate (PET), and/or any other biocompatible absorbable materials as well as other metals besides nickel-titanium alloys, particularly metals having a high strength-to-elastic modulus ratio. The anchor head 29 may be coated, or partially coated, with a biocompatible lubricant that facilitates an easier insertion of the fastener into the body tissue, or prolongs anti-irritant characteristics.

The anchor head 29 may be manufactured using a variety of techniques, including several mass production techniques. For example, stamping techniques, laser cutting techniques and waterjet cutters may be used, among others. In accordance with one illustrative example, the anchor head (29, 129) may be made in two pieces as depicted in FIGS. 2(b)-2(c). Specifically, FIG. 2(b) depicts a top view of an anchor after it is stamped in a manufacturing process, while FIG. 2(c) depicts a schematic elevation cross sectional view of such a process. First, two layers of material 301, 302 (e.g., polymeric sheet material) are disposed on top of each other. Next, a die 310 may cut out a pattern, wherein each of the upper and lower halves of the anchor head are formed from the top and bottom sheets 301, 302, respectively. For example, the upper sheet layer 301 may include legs/struts 3(a, b, c) while the lower sheet layer 302 may include legs/struts 3(d, e, f). The free ends 3′ of each strut may then be attached to each other, such as by welding or other desired attachment technique, for example, by incorporating heating elements 320 into the periphery of the die 310, or by other suitable technique. A mandrel 330 may be disposed between the two layers during the manufacturing operation to impart a curved shape to the anchor such that it maintains that shape in an elastically relaxed state. The mandrel may be removed, for example, when the manufacturing process is complete.

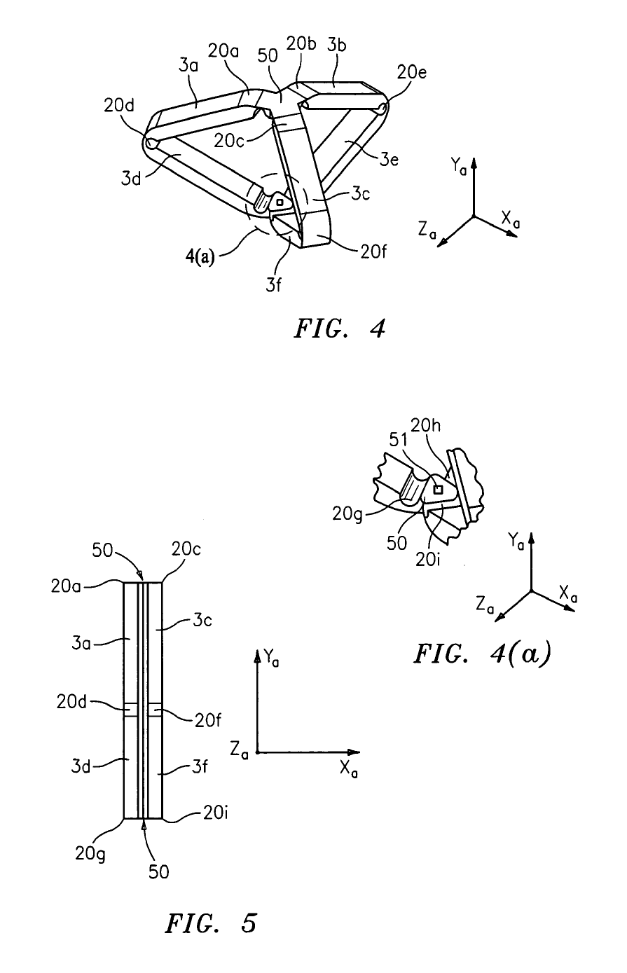

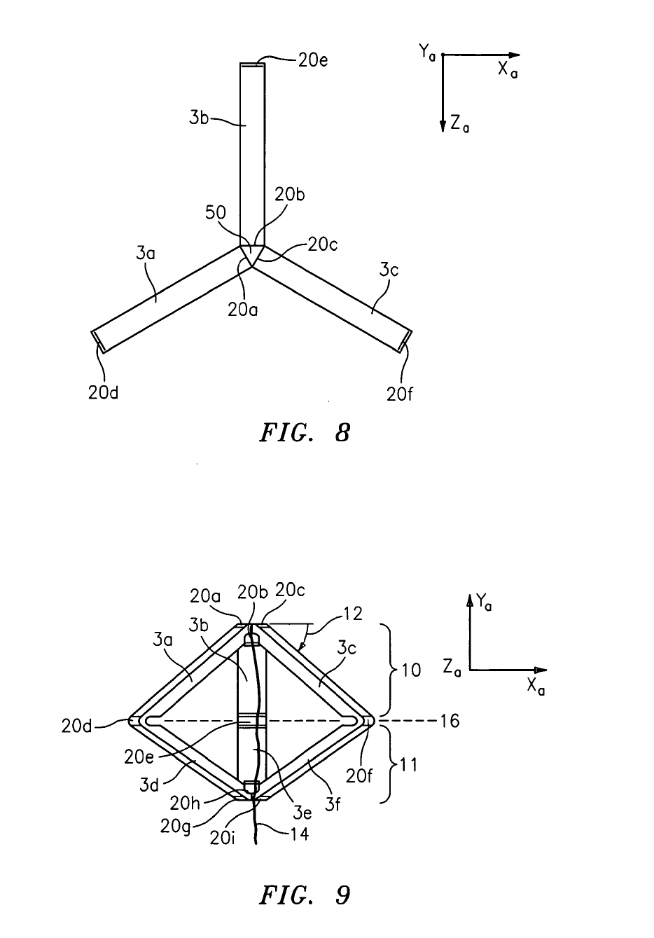

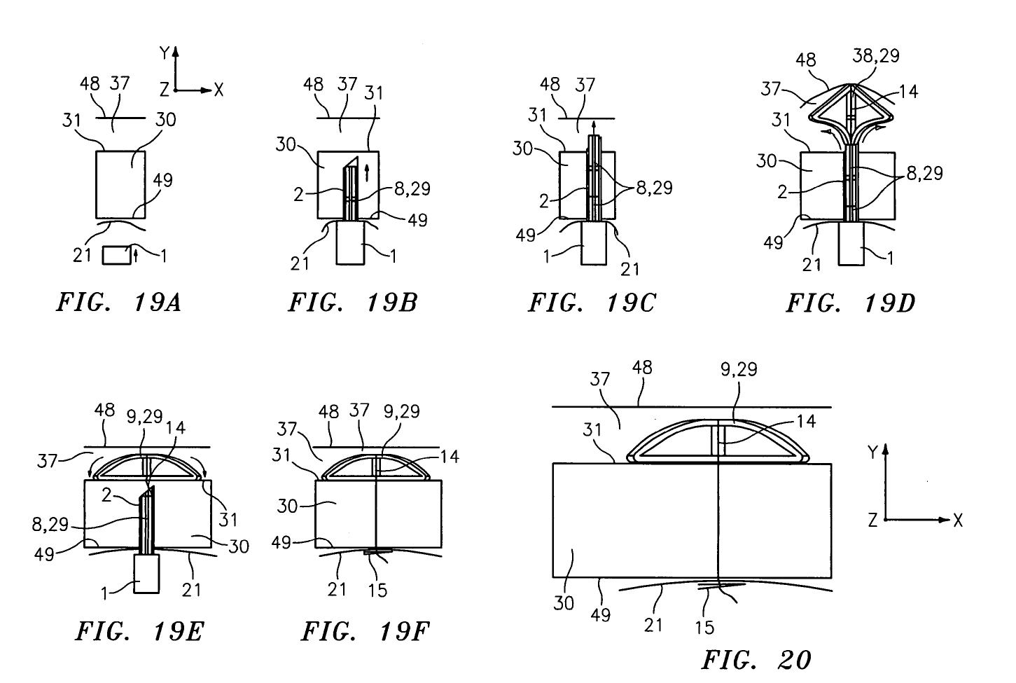

As depicted in FIG. 12, the anchor head 29 is adapted and configured to fit inside an introducer portion 2 of a delivery system with the anchor head 29 in an elongate condition (designated as 8). In a relaxed state, each leg 3a-3f of each anchor head 29 is oriented at an angle 12 with respect to the centerline of the anchor head 29. The orientation of the anchor head 29 is such that the plane of the anchor head base 50 is substantially perpendicular to the longitudinal direction of the introducer 2 of the delivery system. FIGS. 9 and 10 illustrate a filament 14 (e.g., suture thread) that can be embedded in the top section 10 of the anchor head 29 to later facilitate deployment of anchor head 29. Filament 14 serves to connect anchor head 29 to a retainer, such as a suture clip 15, through an opening 51 created by deployment of the suture through inner abdominal fascia 49. Filament 14 may move with the adjacent muscle 30 so as not to tear the muscle 30. While a variety of materials may be used for filament 14, it is preferred to us a standard, non-absorbable suture thread; such as 0 or 2-0 polyethylene or polypropylene suture material. Once the anchor head 29 is deployed and collapsed, as shown in FIG. 7, each leg 3a-f of the anchor head 29 is disposed at an angle 13 with respect to the base plane of the bi-pyramid, such that once fastened to the inner abdominal fascia 49, the bottom section 11 of the anchor head 29 is nearly flush with the anterior fascia 31, as depicted in FIG. 19. The top section 10 of the anchor head 29 preferably possesses a thicker cross-section than the bottom section 11 for the purpose of increased strength and structural stiffness. As further depicted, for example, in FIG. 4, anchor head 29 includes nine living hinges 20a-20i to help maintain compliance between the anchor head base 50 and the muscle layer 30 of patient. The top section 10 of the three legs 3a-c disposes itself in a downward curved position when in the collapsed state 9 as depicted in FIGS. 19(e)-(f) and FIG. 20. This concavity advantageously provides added structural support in the direction of the filament tensile force by making the anchor head 29 more resistant to inversion. Inversion represents a failure mode, wherein the hinges of the legs 3a-f bend such that the angle 13 is negative. The optimized contact geometry is due to the difference in the leg lengths of legs 3a-c with respect to legs 3d-f. During deployment, the living hinges preferably plastically deform to facilitate holding mesh 21 in place.

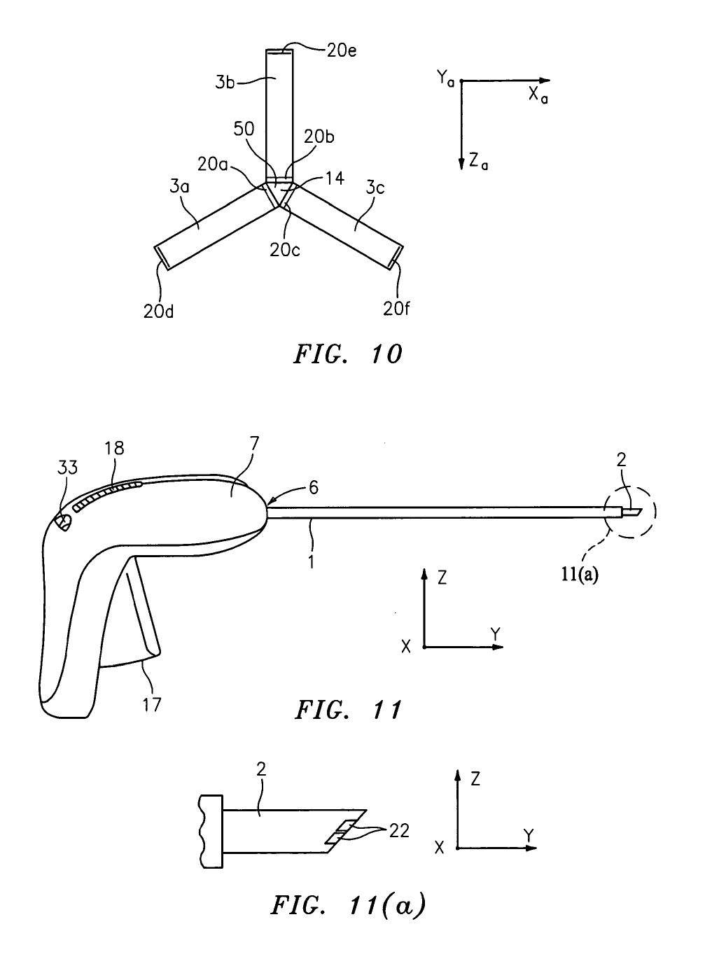

A variety of delivery systems may be used to deliver fasteners in accordance with the present invention. While the delivery system may be reusable, it is preferably a disposable device that may be discarded after a surgical procedure. In order to secure a fastening system including anchor head 29, filament 14 and retainer 15 as embodied herein, as depicted in FIG. 19, it is preferred to insert the anchor head 29 from inside the abdominal cavity through the muscle tissue of the abdominal wall, and then to deploy the anchor in the adipose tissue. Each anchor head 29 is inserted by bringing the distal end of barrel 1 of the delivery system to a desired location on the inside of the abdominal wall of the patient as depicted in FIG. 19(a). This motion also forces the mesh 21 against the interior surface of the abdominal wall. The introducer portion 2 is then extended from the barrel 1 and so as to pierce through the mesh 21 and into the muscle layer 30 as depicted in FIG. 19(b). A sensor 22 is preferably used to detect when the introducer has passed through the muscle and fascia to a location within the adipose tissue 37 below the skin 48 as depicted in FIG. 19(c). During assembly, anchor heads 29 are preferably deformed into the extended state (designated as 8), and then disposed along the length of the barrel 1. The anchor heads 29 are preferably adapted and configured to fit inside an introducer having an inner diameter of about 2-3 millimeters. The introducer preferably has an outer diameter between about 3-4 mm. As depicted in FIG. 11, an actuator 17 may use a mechanical, electromechanical or hydraulic drive, such as a mechanical transmission, such as gears and/or levers (or other means), to actuate a lead screw (not depicted). The lead screw, in turn, is disposed proximally to the anchor heads along the longitudinal axis of the device and advances the anchor heads 29 through the barrel 1 and into the introducer 2 as the actuator 17 is actuated. As the lead screw rotates, the anchor head 29 is pushed out of the tip of the introducer 2. As will be appreciated, the lead screw may be advanced by a totally mechanical means, or the actuator may activate an electrical circuit that drives an electric motor that advances the lead screw.

Once the anchor head 29 has been advanced from the introducer 2 beyond its horizontal mid-plane 16, a ratcheting device inside the delivery device housing 7 can be used to apply tension to the filament 14. The opposite forces applied to the anchor head 29 by the filament 14 (tensile force) and by the other anchors in their extended states 8 while inside the introducer 2 (compressive force) act to compress the anchor head 29 into its collapsed state 9 as depicted in FIG. 19(d). An indicator 18 on the housing 7 (if desired) shows the user when the anchor head 29 is fully deployed.

When the anchor head 29 has been fully deployed against the fascial tissue, the introducer 2 may be extracted from the muscle layer as depicted in FIG. 19(e), as the deployment device maintains the desired tensile force on the filament or suture 14. Retracting the introducer 2 from the inside of the abdominal wall leaves the tail end of the filament 14 visible in the abdomen as depicted in FIG. 19(f). The end of the filament 14 must then be secured with a retainer, such as a suture clip 15 to secure the mesh 21. In accordance with a preferred embodiment, the deployment device is provided with a means for determining, with a high degree of certainty, when the introducer 2 has penetrated the fascial layer 31. This can be accomplished in a variety of manners. For example, electrical impedance measurement, mechanical impedance measurement and optical detection may be used for accomplishing this task. Either way, the sensing means preferably includes a sensor 22 that is located on or next to the introducer cutting surface 5. Differences in the physical (e.g., optical and/or electrical) properties of muscle and adipose tissue may be used to sense the transition from one tissue to the other while the introducer 2 penetrates the fascia between these two tissue layers. Impedance measurement is believed to be a simple and effective method of distinguishing muscle tissue from adipose tissue in vivo. Optical detection may also be used in lieu of or as a compliment to the electrical impedance measurement.

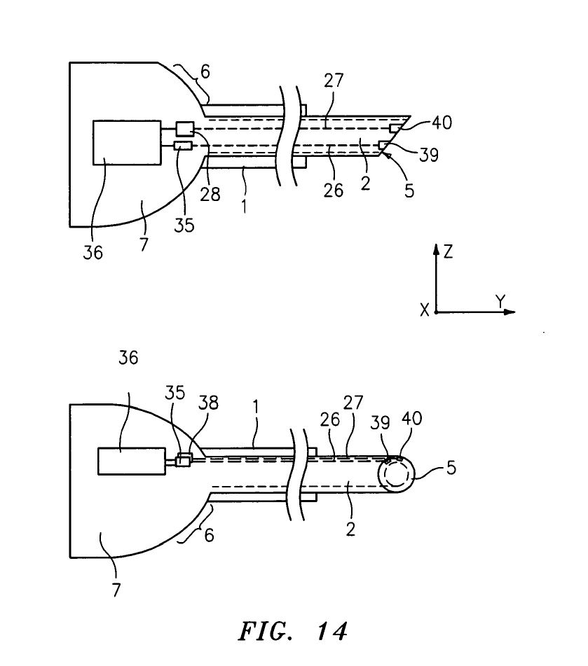

For purposes of illustration and not limitation, as embodied herein, the electrical impedance sensor system can be comprised of two or more electrical contacts that are biocompatible and made of an electrically conductive material. As depicted in FIG. 15, these contacts 23 are preferably positioned proximate the distal end of the introducer 2. Even more preferably, these contacts are flush with the outer curved surface of the introducer 2. As depicted, each contact 23 is electrically insulated from the other when the introducer is outside of the body. The contacts 23 become electrically connected, however, when the introducer is immersed in a conductive substance, such as living tissue rich with fluid. As is further depicted, a wire or other conductor 24 for each contact is embedded within the introducer 2, and runs through the introducer 2 along the length of the barrel 1. As depicted, the wires 24 operably electrically connect the contacts 23 to an impedance measurement circuit 25 in the delivery device housing 7. For example, a Wheatstone bridge or other resistance-measurement circuit may be employed for circuit 25. Circuit 25 is preferably connected to a display device that clearly shows the user either a direct view of the measured impedance or, for clarity, the result of a mapping from said impedance to another scale.

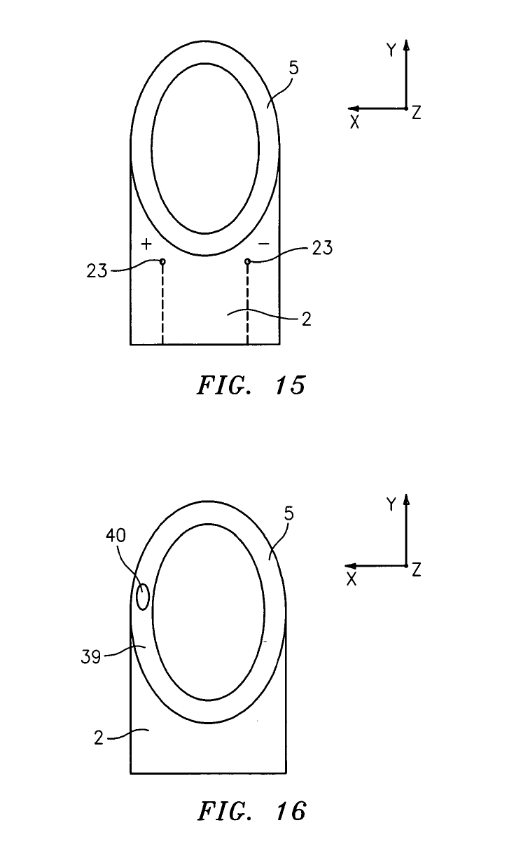

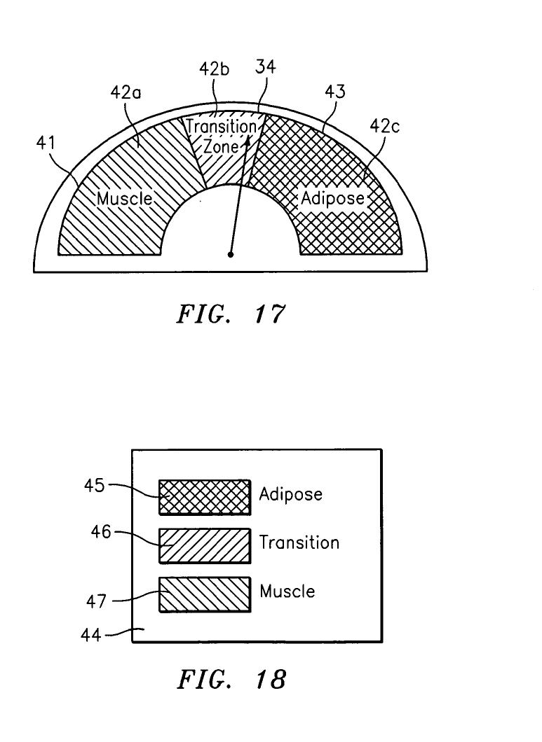

This display may be implemented as a dial indicator, as depicted in FIG. 17, with a gradient and/or threshold sensing level 34, a light or series of lights, as seen in FIG. 18, or other similar graphical user interface. The gauge implementation depicted in FIG. 17 includes a gauge pointer 43 and a gauge face 41. As depicted, the gauge face is divided into at least three sections, including: the indication range for muscle tissue 42a, the indication range for the transition zone 42b, and the indication range for adipose tissue 42c. The threshold sensing level 34 displays the point at which the device senses that the cutting surface 5 is at the desired point of deployment, immediately outside the facial tissue layer 31. The light indicator depicted in FIG. 18 may include an indicator face 44, an adipose tissue indicator light 45, a transition indicator light 46, and a muscle indicator light 47. When the sensor 22 detects the presence of a tissue, the result of this detection is displayed by either lighting the corresponding light on the indicator, in the case of the light indicator of FIG. 18, or moving the gauge pointer 43 to the corresponding indication range, in the case of the gauge display of FIG. 17. The impedance may be measured at any frequency, but certain frequencies may be selected as they are more sensitive to a change from muscle to adipose tissue and vice versa.

For purposes of further illustration, the optical sensor may include one or more light sources 35 as depicted in FIG. 14. The light source or sources may be located, for example, inside the housing 7, immediately proximal to the base of the barrel 6. The light source optical fiber 26 is preferably a thin optical fiber disposed along (or inside of) the wall of the introducer 2 in the axial direction of the barrel 1. The source fiber 26 preferably originates at the base of the barrel 6 and terminates in the source fiber terminator 39 located at the introducer's cutting surface 5, as depicted in FIG. 16. A second optical fiber used for detection 27 is preferably disposed parallel to the source fiber 26, along the curvature of the cross-section of the barrel 1, as depicted in FIG. 14. The detector fiber 27 also spans the distance from the base of the barrel 6 to the cutting surface 5. The distal end of the detector fiber 27 also terminates at the cutting surface 5 of the introducer 2. The source fiber terminator 39 and the detector fiber 40 may include small pieces of plastic, glass, or other translucent, biocompatible material that provides a clear optical interface. The end of the detector fiber 27 within base of the barrel 6 preferably feeds into a photoresistor 28. The photoresistor 28 is positioned next to the light source 35 at the base of the barrel 6. In this embodiment, light from the source must be transmitted through source fiber 26, where it is scattered and filtered by the tissue at 5, before being transmitted back through the detector fiber 27 to the photoresistor 28. Circuit 36 may be used to measure the output of the photoresistor 28 at a given frequency. An indicator on the surgeon-interface casing 7 may directly display the apparent color of the tissue at the cutting surface 5. Alternatively, the circuit 25 may include a mapping or conversion from the apparent tissue color to the probable tissue type at the cutting surface 5. The result of this mapping may be displayed on an indication mechanism, as shown in FIGS. 17 and 18. An analog display, such as seen in FIG. 17, may include a threshold value 34, which indicates the apparent transition from muscle tissue to adipose tissue.

For purposes of further illustration, and not limitation, a second representative embodiment of an anchoring head 129 is depicted in FIG. 21. The embodiment of anchoring head 129 of FIG. 21 is essentially identical to that of FIG. 2(a), with one important difference. Specifically, the legs of top section 10 include extended portions 103 that provide the legs with a longer effective length to help orient the legs during deployment, and to prevent them from moving toward or away from each other when viewed from above. Specifically, the extended portions 103 tend to interact with tissue, making it more difficult for the legs to rotate about the longitudinal axis Y. As is evident, the legs associated with the top section 10 are longer than the legs of the bottom section 11, which is also generally preferred to maintain desirable operation of the anchor head.

The methods and systems of the present invention, as described above and shown in the drawings, provide for a surgical fastener and associated delivery system with superior properties. It will be apparent to those skilled in the art that various modifications and variations can be made in the device and method of the present invention without departing from the spirit or scope of the invention. Thus, it is intended that the present invention include modifications and variations that are within the scope of the appended claims and their equivalents.

1. A surgical fastener comprising a plurality of elongate struts, each strut having a first end, a second end and an intermediate region between the first end and second end, the plurality of struts being connected to each other at the first end and at the second end to form an anchor head, the connection points of the struts cooperating to define a longitudinal axis of the anchor head.

2. The surgical fastener of claim 1, wherein each elongate strut includes a plurality of living hinges adapted and configured to permit the intermediate region of each strut to move toward or away from the intermediate region of the other struts.

3. The surgical fastener of claim 2, wherein each elongate strut includes three living hinges, wherein one living hinge is located proximate each end of the strut, and the third living hinge is located proximate the center of each strut.

4. The surgical fastener of claim 3, wherein the third living hinge is located closer to the second end of each strut than the first end of each strut.

5. The surgical fastener of claim 1, further comprising a filament disposed along the longitudinal axis.

6. The surgical fastener of claim 5, wherein the filament is disposed between the struts.

7. The surgical fastener of claim 5, wherein the filament is attached to the anchor head.

8. The surgical fastener of claim 5, wherein the filament passes through an opening defined at an end of the anchor head.

9. The surgical fastener of claim 5, wherein the anchor head is attached to a first end of the filament, and the fastener further comprises a clip applied to a second end of the filament.

10. The surgical fastener of claim 1, wherein the struts are biased to separate from each other.

11. The surgical fastener of claim 3, wherein each strut includes a first body portion defined between the first living hinge and the third living hinge and a second body portion defined between the second living hinge and third living hinge.

12. The surgical fastener of claim 11, wherein the first body portion of each strut is longer than the second body portion of each strut.

13. The surgical fastener of claim 11, wherein the first body portion of each strut extends beyond the third living hinge to form an extended portion of each first body portion.

14. The surgical fastener of claim 1, wherein the anchor head may be compressed radially inwardly into a generally elongate configuration.

15. The surgical fastener of claim 13, wherein the second body portion of each strut has a smaller cross section than the first body portion of each strut to define a recess to receive the extended portion of each strut when the anchor head is compressed radially inwardly into a generally elongate configuration.

16. The surgical fastener of claim 11, wherein the second body portion of each strut has a smaller cross section than the first body portion of each strut.

17. (canceled)

18. (canceled)

19. The surgical fastener of claim 14, wherein the anchor head is adapted and configured to fit inside a tube having an inside diameter less than about 5 mm when in the generally elongate configuration.

20. The surgical fastener of claim 14, wherein the anchor head is adapted and configured to fit inside a tube having an inside diameter less than about 3 mm when in the generally elongate configuration.

21. The surgical fastener of claim 5, wherein the filament includes a non-absorbable surgical suture.

22. The surgical fastener of claim 1, wherein the anchor head includes a flexible biocompatible material suitable for permanent implantation.

23-45. (canceled)
